(19) United States
(12) Patent Application Publication
(10) Pub. No.: US 2012/0316485 A1
(43) Pub. Date: Dec. 13, 2012

(54) PADDED CYCLING GLOVE THAT REDUCES NERVE INJURY

(76) Inventors: Jason Fryda, Madison, WI (US); Darren Zrubek, Boulder, CO (US); Lori Hoverter, Denver, CO (US); Rhett Griggs, Crested Butte, CO (US)

(21) Appl. No.: 13/493,647

(22) Filed: Jun. 11, 2012

Related U.S. Application Data
(60) Provisional application No. 61/495,845, filed on Jun. 10, 2011.

(51) Int. Cl. A61F 5/00

(52) U.S. Cl. 602/21

(57) ABSTRACT

The present invention relates to a padded glove, used where irritation and injury to the median and/or ulnar nerves in the hand is a common problem. A method and apparatus for protecting nerve regions while bicycling is provided according to various embodiments. The glove padding is located over boney pillars of the palmar-side of the hand in order to direct pressure and/or force caused by the handlebar while cycling or gripping to one or more boney pillars of the hand, rather than over the entire hand and/or over the nerves. The glove padding is located at discrete positions and not broadly over large swaths of the palm.

### CROSS REFERENCE TO RELATED APPLICATIONS

This application claims the benefit of U.S. Provisional Patent Application No. 61/495,845, entitled “PADDED CYCLING GLOVE THAT REDUCES NERVE INJURY” filed on Jun. 10, 2011, the entire disclosure of which is incorporated by reference herein.

### FIELD OF THE INVENTION

This disclosure relates to a padded glove, and methods and apparatus for protecting nerve regions in a user's hands when the user is gripping a solid object.

### BACKGROUND OF THE INVENTION

Irritation and injury to the median (carpal tunnel syndrome) and ulnar (Guyon's canal syndrome) nerves in the hand is a common problem. The nerves pass through separate fibro-osseous canals within the wrist/palm. Nerve injury may occur as a result of compression, vibration, and traction. Common causes of nerve compression include use of a computer keyboard and mouse (mouse use places pressure primarily on the ulnar nerve), and holding a bicycle handlebar. Vibration induced nerve injury may occur from using gardening equipment (i.e., power lawn mowers, gasoline powered blowers, power string trimmers), operating heavy equipment (i.e., back hoes, road graders, tractors), and sports utilizing racquets, bats, or poles. Traction related nerve irritation might arise from frequent repetitive tasks involving hand/wrist motion such as occurs with assembly line work. Many activities may produce nerve irritation or injury through a combination of mechanisms, such as compression and vibration. Examples of such activities include factory work involving the use of hand held power tools, driving/racing cars or boats, piloting aircraft, and prolonged bicycling.

Irritation of the nerves in the hand may present with symptoms of tingling/numbness, pain, and weakness. The anatomical distribution of symptoms is dependent upon the location of nerve injury. Injury to the median nerve in the wrist/palm (i.e., carpal tunnel syndrome) typically involves sensory disturbances involving the thumb, index, long, and radial half ring fingers. Sensory disturbances associated with ulnar nerve injury in the wrist/palm (i.e., Guyon's canal syndrome) typically involve the small and ulnar half ring fingers. Motor involvement can include atrophy of the thenar muscles (i.e., median nerve injury) or hypothenar muscles (i.e., ulnar nerve) resulting in grip and pinch weakness. Pain is usually worse with activity. Night symptoms are a frequent complaint.

Many athletic activities involve prolonged or intense gripping or squeezing of hard objects, which can adversely affect critical nerve regions in the athlete's arm. This may be due to the intense shock, pressure and vibration imparted upon the critical nerves in the athlete's palms. For example, while bicyclist's hands do not support heavy weight, they instead must tightly grip handlebars for long periods of time, perhaps hours during long bicycle rides. One of the problems cyclists face is “Cyclist Palsy” in which compression or stretching of the median and/or ulnar nerves causes tingling or numbness in the fingers. Tennis players, racquetball players and golfers can even experience numbness and weakness in their hands long after they have finished playing. Further, fishermen can suffer hand injury and/or pain caused by gripping a fishing pole and/or during lengthy periods spent reeling-in a catch, during deep-sea fishing. Skiers, in particular cross-country skiers, can suffer injury to their hands from griping ski poles for long periods. Also, motorcyclists or participants in motocross can suffer hand injury or pain from gripping handlebars during long rides.

When people place their hands on a hard object, the primary contacts between the palm of their hands and the surface of the hard object are the hard, bony prominences at the base of the thumb and the lateral side of the hands called the thenar and hypothenar eminences, respectively. These two areas bear much of the pressure and keep the pressure from the object away from the neurovascular bundles. However, under the intense and/or prolonged pressure associated with the gripping process, as is common in the activities described above, these bony prominences separate slightly thereby allowing some of the pressure to be applied to the neurovascular bundles that run between the two bony prominences.

The thenar and hypothenar musculature at the sides of the hands provide a natural protective region to the branches of the medial and ulnar neurovascular region that extend to the athlete's fingers. However, when the hands are wrapped around a hard, cylindrical object, or pressed flat against a solid surface, this natural protective region separates and allows pressure from the object or surface to be applied directly to the medial and ulnar nerve branches. This can result in numbness in the fingers, swelling of the hands, and tennis elbow or carpal tunnel syndrome, which is a variant of ulnar nerve damage due to prolonged exposure to pressure, vibration and the shock of impact.

Pressure exerted by intense gripping restricts the circulation in the neurovascular bundles causing decreased blood flow and a tingling in the hands. Continuous pressure such as in a prolonged bike ride can cause numbness and permanent damage to the user's hands. Further, the athlete's performance can be severely limited from the injury, pain and numbness due to the constant assault on the palm of the hand and the median and ulnar neurovascular complexes caused by intense and prolonged gripping of an object. For a general background on the problems of prolonged or intense gripping or squeezing of hard objects, which can adversely affect critical nerve regions in the athlete's arm, see U.S. Pat. No. 6,845,514 issued to Yao on Jan. 25, 2005 (“Yao”) and U.S. Pat. Pub. No. 2006/0143783 of Berman (“Berman”) published Jul. 6, 2006, the entire disclosures of each of which are incorporated by reference in their entireties.

Various gloves have been designed to mitigate or prevent injury or damage to the nerves as a result of such activities. U.S. Pat. No. 5,404,591 issued to Brinnand et al. on Apr. 11, 1995 (“Brinnand”) discloses a glove with padding around the wrist/carpal area to protect the skin and bones of the hand. The glove is designed for users of electronic input devices such as a computer mouse or stylus. The padding is located across the entire wrist and palm without any recess to off load the median or ulnar nerves.

U.S. Pat. No. 5,921,949 issued to Dray on Jul. 13, 1999 (“Dray”) seeks to protect the median nerve by designing a circumferential compression strap applied around the wrist. Compression by the strap is intended to relax the flexor retinaculum and reverse anterior to posterior prolapse of the carpal tunnel thereby reducing stress on the median nerve. This orthosis does not address direct external pressure to the median and ulnar nerves. It also does not address traction stress to the median and ulnar nerves caused by repetitive wrist motion.

Gloves with pads have been produced to prevent vibration-related nerve injury. Gloves have also been made incorporating bladders to prevent transfer of vibration to the hand. See U.S. Pat. No. 5,771,490 issued Jun. 30, 1998 to Reynolds et al. (“Reynolds I”) and U.S. Pat. No. 5,537,688 issued Jul. 23, 1996 to Reynolds, et al. (“Reynolds II”).

A variety of gloves have been developed for use in sports (See U.S. Pat. No. 4,691,387 issued to Lopez on Sep. 8, 1987 (“Lopez”)) such as racquetball, golf (U.S. Pat. No. 5,634,214 issued to St. Ville on Jun. 3, 1997 (“St. Ville”)), and baseball. These gloves are typically designed to improve a player's grip on the handle of the racquet or bat. Some of these gloves include padding in various areas. But they do not specifically address nerve protection through impact or pressure avoidance to the nerves.

U.S. Pat. No. 5,926,847 issued to Eibert on Jul. 27, 1999 (“Eibert I”) and U.S. Pat. No. 6,216,276 issued to Eibert on Apr. 17, 2001 (“Eibert II”) describe a padded athletic glove wherein pre-formed, air-blown, silicon foam pads are stitched onto the glove, or are applied to the glove using an adhesive. Cycling gloves are mentioned wherein protective padding is provided on the palm and/or fingers of the glove. Athletic gloves are described that protect the palms and/or fingers and/or assist in the catching of a ball or another object and/or enhance the gripping of an object already that is held in the hand. Pads are described that range from about 0.010-inch to about 0.012-inch thick, having a durometer range of about 45 Shore A to about 55 Shore A, and having a specific gravity of about 1.12 to about 1.16.

U.S. Pat. No. 4,747,163 issued to Dzierson on May 31, 1988 (“Dzierson”) provides a glove with resilient padding secured to the palm side of a glove shell, extending from the heel of the palm toward the glove fingers and across the area between the glove index finger and the glove thumb.

U.S. Pat. No. 7,469,426 issued to Roeckl on Dec. 30, 2008 (“Roeckl”) provides a bicycle glove with padding primarily positioned in the region between the thumb crotch and the index finger, in an attempt to relieve pressure spots when gripping bicycle handlebars.

Many gloves designed for cycling provide multiple pads, for example U.S. Pat. No. 6,845,519 issued to Garneau on Jan. 25, 2005 (“Garneau I”), and U.S. Pat. No. 7,578,006 issued to Garneau on Aug. 25, 2009 (“Garneau II”). Each of these disclose gloves intended to increase comfort and/or flexibility, and provide increased breathability, yet do not address protecting the nerves.

U.S. Pat. Pub. No. 2011/0055995 of Dassler (“Dassler”) published Mar. 10, 2011, provides a glove with palm portion formed of flexible material, wherein the palm portion includes multiple ball-shaped elements.

Many bicycling gloves attempt to protect the median and ulnar nerves by providing cushioning directly over the nerves. For example, Minkow, et al (U.S. Pat. No. 6,289,517 issued Sep. 18, 2001) addresses protection of the median and ulnar nerves by placing padding of increased thickness directly over the nerves.

U.S. Pat. No. 6,845,514 issued to Yao on Jan. 25, 2005 attempts to address the problem of irritation/injury to the median and ulnar nerves in the wrist/palm by using pads placed peripherally to the nerves. The objective is to avoid pressure to the nerves rather than to cushion them, so no padding is placed directly over the nerves. However, there is no attempt to direct pressure imparted to the hand to the boney pillars of the hand, therein avoiding or mitigating irritation or injury to the nerves.

The prior art gloves do not adequately protect irritation and injury to the nerves in the hand. The prior art gloves typically provide a broad pad on the palm of the hand or multiple pads assembled primarily on the palm of the hand. Such prior art gloves, however, are not specifically designed for protecting the nerves and/or are not specifically designed so as to direct pressure imparted to the hand to the boney pillars of the hand. Many current padding designs place pads over the ulnar nerve area in hopes that the pressure over the nerve area will be dispersed by the surface area of the pad. Further, the prior art does not provide a glove that, while remaining flexible, avoids pressure to the nerves of the hand, and does not direct pressure to the boney pillars of the hand.

It is therefore desirable to have a glove that provides padding over the boney structure of the hand without compressing one or more nerves, rather than padding over the entire area, therein avoiding or mitigating irritation or injury to the nerves, while also remaining flexible so as to allow gripping. By placing the padding over the boney structure of the hand, the padding functions to transfer pressures and/or forces, such as those generated by gripping a hard surface, to the boney structure of the hand and thus mitigate, if not remove, undesirable transfer of pressures and/or forces to the nerves of the hand, such as the ulnar and/or median nerves.

### SUMMARY OF THE INVENTION

Certain embodiments of the present disclosure relate to a padded glove, and more specifically to a method and apparatus for protecting one or more nerve regions while bicycling. The invention involves the padding of the glove, with particular attention to the location of the padding relative to anatomic structures of the hand. In one preferred embodiment, the padding is located over the boney pillars of the hand in order to direct pressure caused by the handlebar while cycling or gripping to where the hand is meant to carry the pressure (i.e. over the boney pillars), rather than other regions of the hand, in particular over one or more nerves of the hand, where pressure or loading is not intended. The boney pillars comprise any portion of the carpal bones, such as portions of scaphoid bone, trapezium bone, trapezoid bone, capitate bone, hamate bone, pisiform bone, triquetrum bone and lunate bone. Although many of the embodiments are focused on padded gloves for use in cycling, the invention may be used in any application where irritation and injury to the median and ulnar nerves in the hand caused by prolonged pressure applied against these nerves is a common problem. Such applications include athletic activities that involve prolonged or intense gripping or squeezing of hard objects, which can adversely affect critical nerve regions in an athlete's arm. Such other applications include, but are not limited to: motocross riding, motorcycle riding, fishing, golfing, weight-lifting, trek running, trail running, downhill and cross-country skiing, and any application where gripping is required, to include construction such as jack-hammering, clinching of garden tools, and extended clinching of steering wheels.

This is in contrast to the prior art, which has no appreciation for where padding should not be, and generally teaches that the more padding there is the better. Indeed, the prior art teaches away from embodiments of the invention where more padding is not better and in fact may be harmful.

By way of providing additional background, context, the following references are described for the express purpose of explaining the nature of athletic gloves and to further describe the various tools and other apparatus commonly associated therewith:

U.S. Pat. No. 5,987,642 describes a batting glove that includes shock-absorbing vinyl nitril pads (about 1/16 to 3/32-inch thick) that are glued to the glove and then covered by a thin layer of leather sewn to the glove.

U.S. Pat. No. 5,598,582 describes a heat-resistant glove having a raised silicone portion that is formed on the palm by applying the silicone through a nozzle to form a computer-controlled pad pattern, whereupon the silicone is cured.

U.S. Pat. No. 5,790,980 describes a stitched and padded glove that includes a heat softening, slow recovery, medium density, polyurethane foam pad that, when heated by the hand, conforms to the shape of the hand.

U.S. Pat. No. 4,038,787 describes an abrasive glove wherein the palm/fingers of the glove include abrading pads whose upper layer contains grit particles, and whose lower layer is a waterproof adhesive.

U.S. Pat. No. 4,380,093 describes a bicycle handlebar grip that is adapted to fit over the “cross-over” and “turn-of-bar” segments of a racing bicycle handlebar. The grip is shaped to allow a comfortable hand grip in any of several positions. The grip also includes provisions for reducing transmission of vibration from the handlebar to the rider's hand, thus avoiding physical ailments such as “biker's nerve palsy.”

U.S. Pat. No. 5,581,809 describes a protective glove having a number of spaced pads that edge to edge abut when the glove is closed on to an object, so as to form a generally continuous pad for the hand. The use of pads that are made from natural or synthetic rubber, natural or synthetic rubber foams with open or closed cells, polymeric (polyurethane and polystyrene) foams with open or closed cells, or impact absorbent gels that may require a containment pouch, are suggested.

U.S. Pat. Pub. No. 2011/0088139 of Travell (“Travell”) published Apr. 21, 2011 describes a hand protection system for use in sports such as motorcycling. More specifically, Travell provides a system which in use is mounted onto a glove for protecting the back of a user's hand comprising a plurality of elongate, flexible strips, each overlying a respective finger. Each strip has a first end portion serving as sole means of fixedly attaching the strip to the glove, usually in the vicinity of the fingertips, and a remaining portion, extending to the second end of the strip, which is free to move relative to the glove. In this respect, a plurality of discrete guide structures are attached to the glove at a spacing remote from the first end portion) of each strip so that the strip extends across, through or into the guide structure and is held in position on the glove in a manner which allows sliding displacement of the remaining portion of the strip along the longitudinal axis of the strip. The guide structures may be configured to provide a closed tunnel through which the strip extends, or in other versions may be open so that opposing engagement lugs slidingly retain side edges of the strip. Optionally, a plate assembly is mounted over the knuckle and/or metacarpal region of the hand and is configured to provide plural guide portions into which the free second end portions of at least some of the strips are received in slidable manner.

U.S. Pat. No. 7,895,670 issued to Kleinert on Mar. 1, 2011 (“Kleinert”) discloses a glove that flexes as the user clinches his fist. The glove is provided with expandable zones on both sides of the top portion of the glove. Two expandable zones are positioned over the metacarpalphalangeal joints of the one small finger and the index finger. A third expandable zone is positioned over the center axis of rotation of the metacarpalphalangeal joints of the long finger and the ring finger. A cooperating relationship between the three expandable zones allows for expansion of the glove, both longitudinally and laterally, over the metacarpalphalangeal joints of the hand.

The design and application of padding for use in gloves to aid in absorbing impact and/or vibration, and/or to facilitate temperature control (to include cooling and heating) is well-known in the prior art. Such art includes: U.S. Pat. Pub. No. 2009/0035524 of Wyner published Feb. 5, 2009; U.S. Pat. Pub. No. 2010/0154106 of Hammons published Jun. 24, 2010; U.S. Pat. No. 7,254,840 issued to Hammons on Aug. 14, 2007; U.S. Pat. Pub. No. 2009/0307824 of Roeckl published Dec. 17, 2009; and U.S. Pat. No. 5,682,613 issued to Dinatale on Nov. 4, 1997.

According to various embodiments of the present disclosure, one aspect of the invention is to provide a padded glove that protects and/or mitigates nerve injury or irritation by providing one or more pads on a glove that are placed so as to transfer or carry loading through the bony portions of the hand, but wherein the glove is devoid of padding on certain other areas that cover nerve areas of a person's hand, and is flexible to allow gripping. The padded glove comprises one or more pads strategically placed on the palmar portion of glove, that is the underside or palm-side, of the hand.

The phrase “device”, “apparatus”, “glove”, and “padded glove” are used herein to indicate the invention device. The phrase “removably attached” and/or “detachable” is used herein to indicate an attachment of any sort that is readily releasable.

The phrase “boney pillar”, “pillars of the hand”, and “boney pillars of the hand” refer to any portion of any of the bones of the hand in which a force and/or pressure may be imparted, and comprise at least any portion of the scaphoid bone, trapezium bone, trapezoid bone, capitate bone, hamate bone, pisiform bone, triquetrum bone and lunate bone. The phrase “boney pillar” and “pillars of the hand” is not limited to regions and/or portions of any of the bones of the hand that present a protrustion or pillar.

The phrase “carpal bones”, “bones of the carpus”, and “carpus” refer to any portion of one or more of the scaphoid bone, trapezium bone, trapezoid bone, capitate bone, hamate bone, pisiform bone, triquetrum bone and lunate bone.

The phrase “metacarpal bones”, “metacarpals”, “metacarpus” and “bones of the metacarpus” refer to any portion of one or more of the five metacarpal bones.

The phrase “positioned not substantially over the user's median and ulnar neurovascular bundles” means positioned so as to not cover, at any cross-sectional point, more than ten percent of the width or diameter of either of the median or ulnar nerve bundles.

The phrase “positioned not substantially over the user's ulnar neurovascular bundle” means positioned so as to not cover, at any cross-sectional point, more than ten percent of the width or diameter of the ulnar nerve bundle.

In a preferred embodiment of the invention, the device is comprised of a glove that fits over a human hand with lower glove portion and upper glove portion, the glove comprising a main glove body and a glove palm, a finger area and thumb area. The glove may be fitted with glove holes or gaps, for example glove holes on the finger area. In other embodiments, the glove holes may be fitted to the glove thumb area and/or to the main glove body area. The glove may further comprise a closure tab that affixes to the closure tab receiving area. The palmar-side of the glove includes one or more pads located on the glove palm. The one or more glove pads are collectively referred to as “pads” or “glove pads.” The pads may comprise thumb-area pads, side-area pads, and finger-area pads. The pads are deliberately and precisely positioned on the glove to receive pressure, when gripping a hard object such as bicycle handlebars while bicycling, that is directed through the pads and toward the boney regions of the hand, and furthermore, not over broad regions of the hand covering the nerves of the hand.

In a preferred embodiment of the invention, the pads fitted to the glove are of variable height and comprise thumb-area pads, side-area pads, and finger-area pads. In one embodiment of the invention, there are three pads of type finger-area pads, four pads of type side-area pads, and two pads of type thumb-area pads. Further, in one embodiment, each of the three pads of type finger-area pads is of height about 2 mm. In other embodiments, the thickness heights differ by ½ mm or more. Also, in one embodiment, the four pads located in the lower glove area near the wrist of the wearer, are not finger-area pads, rather one is of thumb-area pads and three are of side-area pads; all are of height of about 4 mm. Lastly, the remaining two pads are one of type thumb-area pads and one of type side-area pads; all are of height about 6 mm. In another embodiment, the pads described above as 2 mm in thickness are of 6 mm thickness, and those of 6 mm thickness are instead of 2 mm thickness.

In another embodiment, similar to the embodiment just described, except that those pads of height about 2 mm are of height about 1 mm, those pads of height about 4 mm are of height about 2 mm, and those of height about 6 mm are of height about 4 mm. In another embodiment, the pads described immediately above as 1 mm in thickness are of 4 mm thickness, those of 4 mm thickness are instead of 1 mm thickness.

In another embodiment, all the pads are of the same thickness.

In another embodiment, the pads comprise pads permanently attached. In another embodiment, the pads comprise pads removably attached. The pads may be attached with conventional hook and eye (e.g. Velcro™) loop material, or the like.

In one embodiment of the pads fitted to the palmar side of the glove, the pads comprise a pad surface and pad edge. The pad edge may be of a step-shape, or of more smooth shape, or other shapes that assist in the function of the device in receiving pressure and directing it to the boney regions or members of the hand, and/or designed to assist in attachment of the pads to the glove. In one embodiment of the pads, the pad is domed-shape in cross-section. In another embodiment of the pad, the pad is a dome-shape with a pad depression area. Embodiments of the pads featured in the invention include those of varying cross-sections, comprising rectangular shapes, oval or dome shapes, and those with notches, indentations, contours, ridges, or wavy cross-sections. Also, the pad surface may include those textured and those of varying levels of resilience.

In one embodiment, the shapes of the pads are one or more of substantially oblong, rectangular, or square shape. However, as one skilled in the art would appreciate, the pad cross-section need not be limited to the above shapes. For example, cross-sections of an oval shape or those with at least one defined angle to include obtuse, acute, and right angles can provide a shape in some situations. A substantially round shape may also be employed. Further, shapes specifically or custom-designed for the user may be provided.

Another embodiment of the invention comprises a glove that fits over a human hand with lower glove portion and upper glove portion, the glove comprising a main glove body and a glove palm, a finger area and thumb area. The glove may be fitted with glove holes or gaps, for example glove holes that are designed primarily to provide air circulation and/or distribute perspiration. In other embodiments, the glove holes may be fitted to the glove thumb area and/or to the main glove body area. In one embodiment of the invention, the glove fits over the hand of the user without need of a closure tab which affixes to the closure tab receiving area. The palmar-side of the glove comprises one or more pads located on the glove palm. The pads comprise thumb-area pads, side-area pads, and finger-area pads. The pads are deliberately and precisely positioned on the glove to receive pressure, when gripping a hard object such as bicycle handlebars while bicycling, that is directed through the pads and substantially toward the boney regions of the hand, and furthermore, not over broad regions of the hand comprising the nerves of the hand.

In one embodiment of the invention, the pads fitted to the glove are of variable height and comprise thumb-area pads, side-area pads, and finger-area pads. In this embodiment of the invention, there are four pads of type finger-area pads, four pads of type side-area pads, and two pads of type thumb-area pads. Further, in this embodiment, each of the lowest four pads, located in the lower glove area, are of height about 4 mm. Three of these pads are of type side-area pads and one is of type thumb-area pads. The remaining six pads, are one of type side-area pads, one of type thumb-area pads, and four of type finger-area pads, are of height about 2 mm. In another embodiment, the pads described immediately above as 4 mm in thickness are of 2 mm thickness, and those of 2 mm thickness are instead of 4 mm thickness.

In a preferred embodiment, by virtue of the distinctive shape of pads, substantially located over one or more boney pillars of the palmar-side of the hand, pressure and/or forces caused or imparted by the handlebar while cycling or gripping is directed to the boney pillars of the hand, rather than over the entire hand and/or over the nerves (in particular, the ulnar and/or median nerves). By placing the padding over the boney structure of the hand, the padding functions to substantially transfer pressures and/or forces, such as those generating by gripping, to the boney structure of the hand and thus mitigate if not remove undesirable transfer of pressures and/or forces to the nerves of the hand, such as the ulnar and/or median nerves. The glove padding is located at discrete positions (e.g. to cover a particular carpus bone and/or transfer pressure and/or force primarily to a particular carpus bone) and not broadly over large swaths of the palm. The nerve injury to the wearer is diminished yet hand dexterity and tactile feedback of portions of the hand not requiring padding is preserved.

In one embodiment, a single first pad is located on the palmer-side of the glove so as to receive a gripping force imparted to the user's hand and substantially transfer the gripping force received to at least one of a carpal bone of the user's hand. In particular, the single first pad is positioned substantially over one or more of the carpal bones of the hand, such as the hamate bone and the pisiform bone, and distinctly not positioned substantially over any of the metacarpul bones.

In one embodiment, a single first pad is positioned on the palmar-side of the glove body to maintain a gap between an edge of the single first pad and an edge of the user's palm.

In another embodiment, in addition to the immediately-above single first pad, a second pad is additionally positioned on the palmer-side of the glove so as to receive a gripping force imparted to the user's hand and substantially transfer the gripping force received to at least one of a carpal bone of the user's hand. In this embodiment, the first pad and the second pad may additionally be configured so as to be in communication when a gripping force is imparted to the user's hand such that the first pad and the second pad, in combination, substantially transfer the respective gripping force received to at least a plurality of carpal bones of the user's hand. Optionally, in this embodiment, the first pad and the second pad may be configured to form a gap between an edge of the first pad and an edge of the second pad when the user's hand is at least one of substantially unflexed, substantially relaxed and not gripping, and further the first pad and the second pad may be configured to substantially close the gap when the user's hand is at least one of gripping and forming a fist. In one embodiment, the first pad is positioned substantially over the hamate or pisiform bone so as to transfer gripping force received by the first pad to the hamate or pisiform bone, and the second pad is positioned substantially over the scaphoid or trapezium or trapezoid bone so as to transfer gripping force received by the second pad to the scaphoid or trapezium or trapezoid bone. In one embodiment, each of the first and second pad are positioned on the palmar-side of the glove body to maintain a gap between a respective edge of the pads and an edge of the user's palm.

In one embodiment of the invention, the pads are located no less than about 2 mm from any nerve regions of the hand. In another embodiment, the pads are located such that when woren by the user, the pads do no impart any significant pressure of force to any region of nerves in the hand.

In another embodiment, the interstitial spaces between such pads is occupied by one or more of: a void or space, a bladder that may be filled with a substance to include water, air, gel, ice, and resilient material. The resilient material may be of uniform or non-uniform resilience. The resilient material may be temporally stable or static or it may vary with time or condition or user input. In one embodiment, the one or more pads are considerably harder than conventional gel or foam cushion elements; a hardness value over 20 Shore A for the pads may provide acceptable comfort and durability. In one embodiment, the hardness range for the pads is between 30 Shore A and 80 Shore D. In another embodiment, a neoprene shaped pad having hardness of 87 Shore A is used. In one embodiment, the one or more pads are substantially flat, however it is also possible that they have an elliptical shape or a hemispherical shape. It is also possible that the pads can be pyramid shaped, triangular shaped, cube shaped, cylinder shaped, trapezoid shaped, parallelepiped shaped, tube shaped, bean shaped, capsule shaped or box shaped.

In other embodiments of the invention, the pads and/or glove comprise temperature, humidity, perspiration, and moisture control elements, such as to cool or heat the user's hands while wearing the gloves. Such temperature control elements include those with active heating or cooling, for example through electrical means, and passive heating or cooling, such as through air venting ports, holes, and channels. In one embodiment, temperature is controlled through chemical means. In another embodiment, temperature control is enabled through evaporative heating. The means to provide temperature, humidity, perspiration, and moisture control are fitted to the glove on any portion of the glove to include the palmar side of the glove, the distal side of the glove, lateral sides of the glove, and any combination therein.

In one embodiment of the invention, the stiffness of the pads is variable. For example, the upper or outer section of the pad may be of higher or lower stiffness than the inner or lower section of the pads. The stiffness of the pads may vary in the longitudinal axis, lateral axis, or combination thereof. The stiffness may vary with time, temperature, or through a user-initiated manner, such as by varying the pressure of air within a pad when air is used within the pad. The stiffness may be tuned or adapted to a particular user.

In one embodiment of the invention, the pads include a component that provides an arch-like support conformal to the user hand anatomy.

In one embodiment of the pads, the user may inject a substance into the pads, such as air, water, and a gel, for user-determined stiffness and/or user preference. The pads may also be specialized for a particular user, for design or aesthetics, or to employ gels and/or padding typically used in athletic footwear, such as monofilaments.

In one embodiment of the invention, the pads are configured such that when the palm is open, the pads comprise distinct pad regions, but when the user clinches their hand, such as when gripping a bicycle handlebar, the pads move together to effectively form a substantially uniform pad. In one embodiment, when the user grips a handlebar or clinches his fist, the pads draw together so as to leave little or no space between pads. In another embodiment, when the user grips a handlebar or clinches his fist, the pads draw together so as to leave a channel or gap below the surface of the pads yet on their exterior surface (i.e. that which touches and engages the handlebar) the pads provide no space between pads.

In one embodiment of the invention, finger-area pads are configured such that when the user clinches their fist and/or grips an object such as a bicycle handlebar, the finger-area pads engage one another so as to form a substantially continuous top or upper surface yet maintain a channel between the substantially continuous upper surface and the glove. Such an embodiment allows, for example, air flow through the channel.

In another embodiment of the invention, finger-area pads are configured that, when the user clinches their fist and/or grips an object such as a bicycle handlebar, the finger-area pads engage one another so as to form a substantially continuous top or upper surface and substantially align laterally such that no gap or channel is formed.

Embodiments of the pads that combine features of the two embodiments described immediately above are included in the invention. For example, embodiments that comprise one or more pads that provide a channel and other pads that do not provide a channel, and combinations that provide or do not provide channels distributed among the various pad types discussed above, to include thumb-area pads, side-area pads, and finger-area pads. Also, some embodiments provide more than one channel below the surface of engaged pads.

In another embodiment of the invention, the pads are overlaid with a protective cover or plate. This embodiment is of particular use when the invention is used in the motocross or motorcycle application, where hardened plates are commonly provided as components of gloves. Such plates may be permanently attached or removably attached. Such plates may comprise materials of metallic substances, alloys, composites, and plastics. The protective cover or pad may comprise multiple pieces or sections, or be of one piece. The protective pad or cover may be substantially plate and/or two-dimensional, or of three-dimensional shape, such as a generally curved shape. The protective pad or cover may have design or aesthetic features, and may include contours, ridges. Further, the protective pad or cover may comprise multiple materials or be of uniform material.

In one embodiment of the invention, the glove has only one pad comprising a location substantially in the finger area of the palm of the glove, the side or lateral area of the palm of the glove and the thumb area of the palm of the glove. In another embodiment of the invention, the glove has a plurality of pads comprising locations substantially in the finger area of the palm of the glove, the side or lateral area of the palm of the glove, the thumb area of the palm of the glove. In another embodiment of the invention, the glove has two pads comprising a location substantially in the finger area of the palm of the glove, the side or lateral area of the palm of the glove, the thumb area of the palm of the glove. In another embodiment of the invention, the glove has three pads comprising locations substantially in the finger area of the palm of the glove, the side or lateral area of the palm of the glove, the thumb area of the palm of the glove. In another embodiment of the invention, the glove has one or two pads comprising a location substantially in the finger area of the palm of the glove, the side or lateral area of the palm of the glove, the thumb area of the palm of the glove. In other embodiments of the invention, the pads are of quantity and placement of any combination of the above.

In another embodiment, a fingerless glove is fitted with padded portions. The gloves may also include partial finger-length fingers, such as ¾-length fingers or ½-length fingers. It would be apparent to one of ordinary skill in the art that a full-figured glove could also be used. Cushions or pads (i.e. padded portions) can be implemented as any of a variety of conventional padding material such as foam rubber of varying densities and thicknesses, layers of fabric of various types and thicknesses, conventional gel or plastic material, an inflatable air-retaining or liquid-retaining vessel, or other types of conventional materials for dissipating pressure across a large surface area. Pads are sewn into the fabric of a fingerless glove. Alternatively, pads may be integrated into the glove as a pocket that may be later stuffed or filled with cushioning material. In another alternative embodiment, pads may be affixed to a fingerless glove with a bonding agent or adhesive material suitable for application to a glove. In yet another alternative embodiment, pads may be affixed to a fingerless glove with conventional hook and eye (e.g. Velcro™) strips thus providing an embodiment with removable pads. In other embodiments, pads may be fabricated from rigid materials such as plastics, fiberglass, or metal materials. It will be apparent to one of ordinary skill in the art that many other implementations of the pads may be used according to the present invention.

In another embodiment, the pads are composite pads comprised of multiple layers of cushioning material. For example, this cushioning material is comprised of foam rubber material of various densities as commonly available. Alternatively, the composite pad may be fabricated from fabric layers, pliable plastic material, sponge, or other soft cushioning material. Layers may be affixed to other layers using conventional bonding agents such as glues. Also, voids may be inserted between layers. It will be apparent to one of ordinary skill in the art that various layers may be fabricated from any of a variety of cushioning materials other than the conventional gel material used in this embodiment.

The invention finds general utility in athletic gloves, including gloves in which padding and/or protection is provided for only the back of the glove, or gloves in which padding and/or protection is provided for both the back and the palm of the glove. In one embodiment, athletic gloves in the form of cycling gloves comprise a palm side that is formed of a perforated or breathable synthetic leather, a back side that is formed of an elastic textile material, for example a polyurethane elastic textile such as Spandex or Lycra, and a wrist-encircling portion that includes a hook and loop closure member such as Velcro.

Cycling gloves having palm pads in accordance with the invention aid a cyclist in gripping the handlebar of a bicycle, as the glove palm pads support, protect, dampen and absorb shock, and pad the cyclist's hands, and as the glove palm pads minimize the occurrence of numbness of the hands of a cyclist.

When athletic gloves constructed and arranged in accordance with the invention are worn, the upper or synthetic leather layer of each of the palm pads is exposed, and this upper layer aids in the gripping of an object such as the handlebar of a bicycle.

As used herein, the term foam or foam layer means a flexible, porous, natural or synthetic material whose volume contains a relatively high percentage of open or closed cells, and a relatively low percentage of the natural or synthetic material from which the foam is formed. The term foam or foam layer, as used herein, is also intended to include a foamed or expanded plastic material that has been treated so as to cause air or gas bubbles or cells to be formed therein, these cells being, either closed cells or open cells. However, within the spirit and scope of the invention, the term foam or foam layer includes both presently known and after-developed equivalent means, such as gel-filled members and/or liquid-filled members. A non-limiting example is an open cell polyurethane foam. While the invention will be described while making reference to the use of foam that may include open cell and/or closed cell foams, the spirit and scope of the invention includes the use of both existing equivalent foam members and after-developed equivalent foam members. Examples of existing and equivalent foam members include, but are not limited to, gel-filled foam members, liquid-filled foam members, air-filled foam members, and combinations thereof.

The pads may be made of a variety of materials, such as foamed material, gel, latex, rubber or mixtures or combinations thereof, to include those described in U.S. Pat. No. 6,618,860 and U.S. Patent Pub. No. 2007/0113317, both of which are herein incorporated by reference in their entireties. The pad material may include gels known to those skilled in the art of gel paddings used in athletic equipment. Further, the pads may include a partly compressible material for added comfort, for example, open cell and/or closed cell foams. A person skilled in the art will appreciate that the use of both existing equivalent foam members and after developed equivalent foam members. Examples of existing and equivalent foam members include, but are not limited to, gel-filled foam members, liquid-filled foam members, air-filled foam members, memory foam members, bio-gel members and combinations thereof.

The term foam or foam layer, as used herein, is also intended to include a foamed or expanded plastic material that has been treated so as to cause air or gas bubbles or cells to be formed therein, these cells being, either closed cells or open cells. However, within the spirit and scope of the invention, the term foam or foam layer includes both presently known and after-developed equivalent means, such as gel-filled members and/or liquid-filled members. A non-limiting example is an open cell polyurethane foam. While the invention will be described while making reference to the use of foam that may include open cell and/or closed cell foams, the spirit and scope of the invention includes the use of both existing equivalent foam members and after developed equivalent foam members. Examples of existing and equivalent foam members include, but are not limited to, gel-filled foam members, liquid-filled foam members, air-filled foam members, and combinations thereof.

In accordance with the invention, the selective application of heat and line pressure to the pads may operate to adhesively attach the pads to the palm of an athletic glove, in embodiments where the pads are permanently attached. In one embodiment of the invention, the relatively thick foam layer within each pad is permanently melted and cut by the application of heat and pressure to a thin layer, to thereby form thin depression lines within each of the pads. In another embodiment of the invention each pad includes internal pad areas provided with a number of relatively thick foam layers that individually define the individual internal pad areas, and the application of heat and pressure operates to secure the pad's upper layer without melting the relatively thick foam layers that define the individual pad areas. Internal depression lines that are formed in one or more of the palm pads operate to divide the palm pad(s) into a number of smaller size and internal pad areas. The thickness of each pad area is equal in thickness to the sum of the upper synthetic leather layer, the uncompressed thickness of the middle foam layer, and the thickness of the adhesive layer. In one embodiment, each of the embossed pad areas has a thickness that is only slightly greater than the uncompressed thickness of the foam layer.

In accordance with the invention, each pad includes one continuous depression line that encircles the edge of each pad, so as to provide that the edge of each pad is continuously sealed to the palm of the glove by the application of heat and pressure. However, within the internal area of each pad, a desired pattern of inner depression lines may be provided to suit the flexure needs of a particular area of the glove palm, including depression lines within a pad that are linear or curved, and depression lines within a pad that cross each other as they traverse the pad.

As a feature of the invention, prior to the above-described application of heat and line pressure, each of the above-described pads may be formed into a unitary three-layer assembly, with this unitary assembly then being adhesively attached to the palm of an athletic glove by the selective application of heat and line pressure. As a feature of the invention, pads in accordance with the invention include a bottom heat-sensitive adhesive layer and a top layer of a synthetic leather that have generally the same planar shape and size, and a middle foam layer that is of generally the same planar shape, but is of a somewhat smaller size. The top and bottom layer are arranged in general positional coincidence, and the middle layer is centered between the top and bottom layer, so that the circumferential edge of the pad comprises only the top and bottom layer. In a first embodiment of the invention the above-described middle foam layer comprises a single piece of material. In a second embodiment of the invention the above-described middle foam layer includes a number of individual foam pieces, one piece for each individual pad area that is to be formed internal of the pad.

Within the spirit and scope of the invention, the above-described pads may be adhesively attached to the palm of an athletic glove only at the location of the above-described heat and pressure, or alternatively the entire area of the pad adhesive layer may be activated by heat, while only the depression line portion of a pad is subjected to both heat and pressure.

In addition, and as a feature of the invention, the pads may be “tacked” to the palm of an athletic glove, using heat in the absence of pressure, and the above-described heat and line pressure may thereafter be applied to the pads and the athletic glove having the pads “tacked” thereon.

While the term synthetic leather is used herein, it is intended that the use of a more expensive natural leather, rather than a synthetic material that is fabricated so as to imitate natural leather, is also encompassed by the term synthetic leather.

In one embodiment of the invention, the body of the glove can be made of, for example, a mesh material or a moisture-wicking material. Additional layer(s) of leather or leatherette material are placed in the thumb-index web space to enhance wear properties of the glove. The materials used for the body of the glove can be of varying colors and patterns to enhance the visual appearance of the glove and to suit personal preference. The pads may be sewn into the palmar aspect of the body of the glove between a thinner layer of material inside the glove and the thicker, more durable leatherette outer material.

In one embodiment of the invention, a strap may be present at the lower portion of the glove arising from the radial side of the body of the glove. Velcro™ loop material, or the like, may be attached to the undersurface of this strap. Velcro™ hook material or the like may be attached to the proximal surface of the ulnar portion of the glove body to co-act with the Velcro™ loop material and create a means by which the strap can be adjustably secured to the body of the glove. The glove can include insulating, heat retaining materials such as Thinsulate™.

In one embodiment, some of the pads are of 2 mm thickness, some of 4 mm thickness, and others of 6 mm thickness. In another embodiment, some of the pads are of 2 mm thickness and others are of 4 mm thickness. In another embodiment, all the pads are of the same thickness.

Stated another way, the average thickness of the one or more pad components typically ranges from about 1-10 mm, even more typically from about 2-8 mm, and even more typically from about 2-6 mm.

In one embodiment, the pads are not of substantially uniform thickness, but instead form contours, ridges, or patterns.

While various configurations of the glove are herein specified, this description in only exemplary and is not intended to limit or otherwise narrow the invention. The glove can include any number of layers in any potential combination thereof as desired for achieving the comfort properties and padding provided by the glove. Further, it shall be understood that the glove may be formed from layers, such layers may individually be formed of a uniform, monolithic material construction or, alternatively, such layers can themselves be composed of a plurality of material layers.

It will also be understood that the finger portions of the glove can be cut anywhere along the length of cyclist's fingers. While cycling hand gloves are shown as having short fingers, a person skilled in the art will appreciate that the cycling hand glove can be long finger gloves, including hand gloves that are constructed and arranged for use in winter temperatures.

One skilled in the art will appreciate that the glove and its features, to include its pads, need not be limited to those specific embodiments described above. Other forms, shapes or designs that enable the foregoing aspects of the present invention are hereby incorporated into this disclosure. Forms, shapes and designs that relate to the padding of the boney portion of a hand to enable pressure to be substantially loaded to the boney portion of the hand are considered to be within the scope of the present disclosure.

In one embodiment of the invention, the location of the pads is identified with reference to specific boney regions of the hand, so as to direct pressure and/or force substantially through the one or more pads and in-turn substantially to the one or more boney regions or bones of the hand. In one preferred embodiment, the boney regions of the hands that receive pressure and/or force comprise the pisoform, hamate, scaphoid, and trapezium bones.

In one embodiment, the one or more pads are positioned so as to engage or receive/transfer pressure/force from the pillars of the arching carpal bones and not positioned substantially over one or more nerves of the hand.

Although the invention has been described with application to gripping, the invention also finds application to leaning or other load-bearing activities. For example, when a bicycle rider leans or pushes against bicycle handlebars for extended periods rather than grips the handlebar or alternately grips and leans against the handlebar.

One of ordinary skill in the art will appreciate that embodiments of the present disclosure may have various sizes. The sizes of the various elements of embodiments of the present disclosure may be sized based on various factors including, for example, the anatomy of the user or cyclist, and the device being handled by the user such as a bicyclist.

One of ordinary skill in the art will appreciate that embodiments of the present disclosure may be constructed of materials known to provide, or predictably manufactured to provide the various aspects of the present disclosure. For example, materials used in the interior and/or exterior of the pads may include, for example, rubber, latex, synthetic rubber, and other fiber-encased resinous materials, synthetic materials, polymers, and natural materials.

This Summary of the Invention is neither intended nor should it be construed as being representative of the full extent and scope of the present disclosure. The present disclosure is set forth in various levels of detail in the Summary of the Invention as well as in the attached drawings and the Detailed Description of the Invention, and no limitation as to the scope of the present disclosure is intended by either the inclusion or non-inclusion of elements, components, etc. in this Summary of the Invention. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below. However, the Detailed Description of the Invention, the drawing figures, and the exemplary claim set forth herein, taken in conjunction with this Summary of the Invention, define the invention.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures.

FIG. 1A is an outline-view of the dorsal-side of a glove of the invention;

FIG. 1B is a top-view of the palmar-side of the glove of FIG. 1A;

FIG. 2A is a cross-sectional view of Section A-A of FIG. 1B, detailing a cross-section of one of the side-area pads;

FIG. 2B is a cross-sectional view of Section A-A of FIG. 1B, detailing an alternate embodiment of a cross-section of one of the side-area pads;

FIG. 2C is a cross-sectional view of Section A-A of FIG. 1B, detailing an alternate embodiment of a cross-section of one of the side-area pads which includes a pad plate feature;

FIG. 2D is a cross-sectional view of Section B-B of FIG. 1B, detailing a cross-section of three of the finger-area pads when the glove is worn by a user not gripping and with the user's hand flat;

FIG. 2E is a cross-sectional view of Section B-B of FIG. 1B, detailing a cross-section of three of the finger-area pads in the embodiment of FIG. 2D when the glove is worn by a user who is gripping an object;

FIG. 2F is a cross-sectional view of Section B-B of FIG. 1B, detailing a cross-section of three of the finger-area pads in an alternate embodiment when the glove is worn by a user not gripping an object and with the user's hand flat;

FIG. 2G is a cross-sectional view of Section B-B of FIG. 1B, detailing a cross-section of three of the finger-area pads in the embodiment of FIG. 2F when the glove is worn by a user gripping an object;

FIG. 3 is a top-view of the palmar-side of an alternate embodiment of a glove of the invention.

FIG. 4A is a schematic anatomical view of selected bones and nerves of the human hand from the palmar side; and

FIG. 4B is a schematic anatomical view of selected bones of the human hand from the palmar side.

The drawings are not necessarily drawn to scale.

### DETAILED DESCRIPTION

The present invention relates to a padded glove, used in any application where irritation and injury to the median and ulnar nerves in the hand is a common problem. Such applications include athletic activities that involve prolonged or intense gripping or squeezing of hard objects, which can adversely affect critical nerve regions in the athlete's arm. One such application is cycling. The glove apparatus invention involves the padding of a glove, with particular attention to the location of the padding relative to anatomic structures of the hand. The glove padding is located substantially over boney pillars of the palmar-side of the hand in order to direct pressure caused by the handlebar while cycling or gripping to the boney pillars of the hand, rather than over the entire hand and/or over the nerves. The glove padding is located at discrete positions and not broadly over large swaths of the palm.

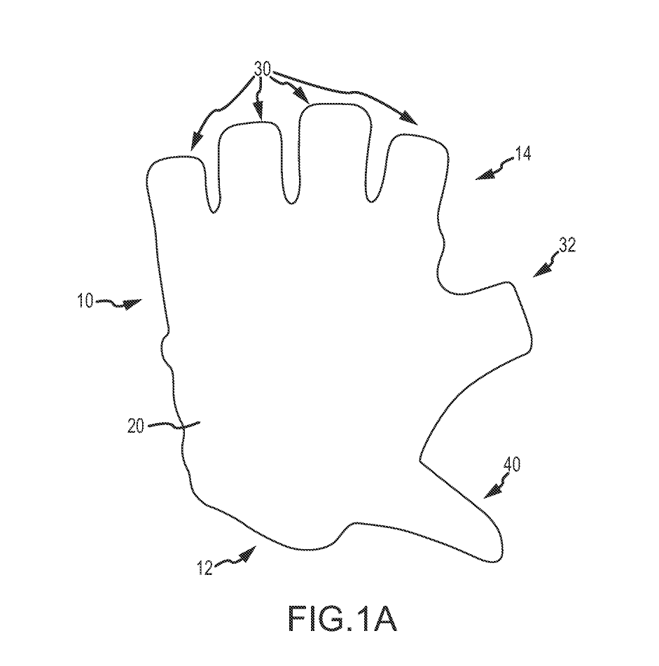

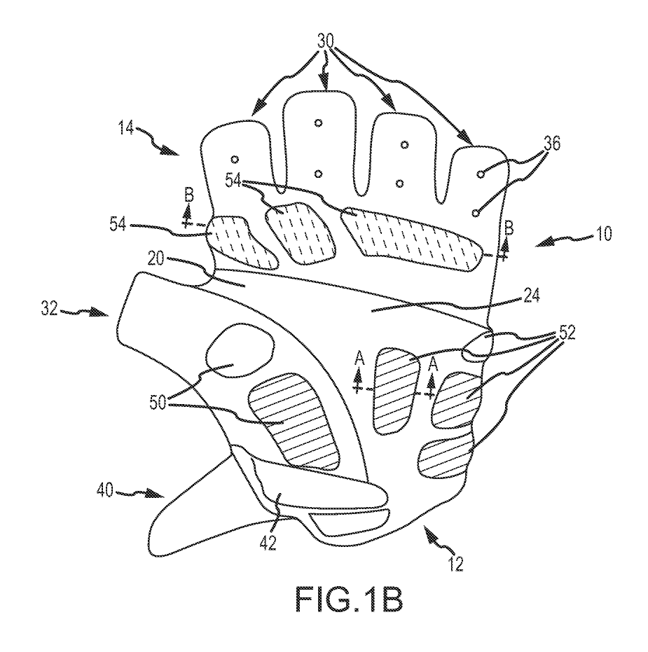

Referring now to FIGS. 1-3, several embodiments of the present invention are shown. In regard to FIGS. 1A-B, top-views of the glove are provided. FIG. 1A is a top-view of the dorsal-side of the glove, and FIG. 1B is a top-view of the palmar-side of the glove. Thus, as shown in each of FIGS. 1A and 1B, a glove 10 that fits over a human hand is shown with lower glove portion 12 and upper glove portion 14, the glove 10 comprising a main glove body 20 and a glove palm 24, a finger area 30 and thumb area 32. The glove 10 may be fitted with glove holes 36 or gaps, for example, glove holes 36 as shown in FIG. 1B on the finger area 30. In other embodiments, the glove holes 36 may be fitted to the glove thumb area 32 and/or to the main glove body area 20. The glove 10 may further comprise a closure tab 40 which affixes to the closure tab receiving area 42. The palmar-side of the glove 10 as shown in FIG. 1B depicts one or more pads located on the glove palm 24. The one or more glove pads are collectively referred to as “pads” or “glove pads.” The pads comprise thumb-area pads 50, side-area pads 52, and finger-area pads 54. The pads are deliberately and precisely positioned on the glove 10 to receive pressure, when gripping a hard object such as bicycle handlebars while bicycling, that is directed through the pads and substantially toward, and substantially received by, the boney regions of the hand, and furthermore, not over broad regions of the hand comprising the nerves of the hand.

In the embodiment of the invention of FIGS. 1A-1B, the pads fitted to the glove 10 are of variable height and comprise thumb-area pads 50, side-area pads 52, and finger-area pads 54. In this embodiment of the invention, there are three pads of type finger-area pads 54, four pads of type side-area pads 52, and two pads of type thumb-area pads 50. Further, in this embodiment, each of the three pads of type finger-area pads 54 are of height about 2 mm. Also, the four pads of located in the lower glover 12 area that are not of type finger-area pads 54 and are of solid-line cross-hatch (one of which is of type thumb-area pads 50 and three of which are of type side-area pads 52) are of height of about 4 mm. Lastly, the two pads shown in FIG. 1B without any cross-hatching (one of type thumb-area pads 50 and one of type side-area pads 52) are of height about 6 mm.

In another embodiment, the embodiment of FIG. 1B is identical to the embodiment just described, except that those pads of height about 2 mm are of height about 1 mm, those pads of height about 4 mm are of height about 2 mm, and those of height about 6 mm are of height about 4 mm.

In another embodiment, all the pads are of substantially the same thickness.

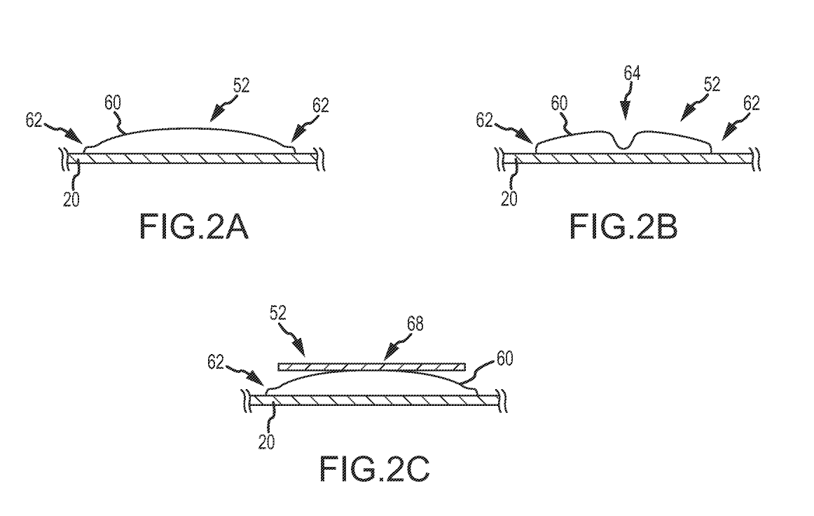

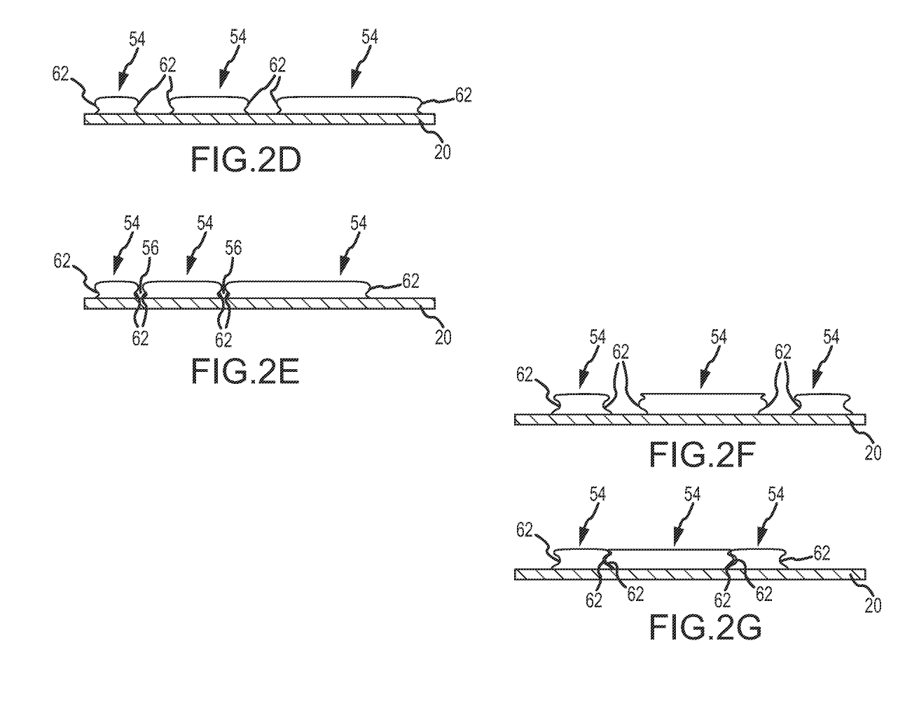

Referring now in detail to FIGS. 2A-2C, a cross-section of one of the pads of FIG. 1B, shown as section A-A, is depicted. The pad is of type side-area pads 52. Each of FIGS. 2A, 2B and 2C depict a pad with pad surface 60 and pad edge 62. The pad edge 62 may be of a step-shape, as depicted in FIG. 2B, or of more gradual shape, as depicted in FIG. 2A. The embodiment of FIG. 2A provides a domed-shape for the cross-section of the pad. The embodiment of FIG. 2B provides a dome-shape with a pad depression area 64. The embodiment of FIG. 2C provides a domed-shape for the cross-section of the pad, similar to that of FIG. 2A, yet includes a pad plate 68. The pad plate is of particular use when the invention is used in the motocross or motorcycle application, where hardened plates are commonly provided as components of gloves. Such plates may be permanently attached or removably attached.

Referring now in detail to FIGS. 2D-2G, cross-sectional views of Section B-B of FIG. 1B of three finger-area pads 54 are provided.

FIGS. 2D and 2E depict one embodiment of the finger-area pads 54 that, when the user clinches their fist and/or grips an object such as a bicycle handlebar, the finger-area pads 54 engage one another so as to form a substantially continuous top or upper surface yet maintain a channel 56 between the substantially continuous upper surface and the glove body 20, as shown in FIG. 2E. FIG. 2D depicts an embodiment in which the user is not clinching their fist and/or gripping an object but rather maintains their hand in a flat position. Such an embodiment allows, for example, air flow through the channel 56.

FIGS. 2F and 2G depict an alternate embodiment of the finger-area pads 54 that, when the user clinches their fist and/or grips an object such as a bicycle handlebar, the finger-area pads 54 engage one another so as to form a substantially continuous top or upper surface and substantially align laterally such that no gap or channel is formed, as shown in FIG. 2G. FIG. 2F depicts the embodiment when the user is not clinching their fist and/or gripping an object but rather maintains their hand in a flat position.

Embodiments of the pads that combine features of the two embodiments described in FIGS. 2D-2G are included in the invention. For example, embodiments that comprise one or more pads that provide a channel 56 and other pads that do not provide a channel 56, and combinations that provide or do not provide channels distributed among the various pad types discussed above, to include thumb-area pads 50, side-area pads 52, and finger-area pads 54. Also, some embodiments provide more than one channel below the surface of engaged pads.

Although FIGS. 2A-2G depict a cross-section of side-area pads 52 and the above discussion describes features associated with interaction between side-area pads 52, the features and functions also apply to combinations of different types of pads. That is, finger-area pads 54, side-area pads 52, and thumb-area pads 50 may be configured to cooperatively engage as a function of the user gripping a handlebar. Specifically, a side-area pad, such as the side-area pad 52 in FIG. 1B from which the cross-sections of FIGS. 2D-2G is provided, may combine with one or more of the thumb-area pads 50.

Embodiments of the pads featured in the invention include those of varying cross-sections, comprising rectangular shapes, oval or dome shapes, and those with notches, indentations, contours, ridges, or wavy cross-sections. Also, the pad surface 60 may include those textured and those of varying levels of resilience.

In one embodiment, the shapes of the pads are one or more of substantially oblong, rectangular, or square shape. However, as one skilled in the art would appreciate, the pad cross-section need not be limited to the above shapes. For example, cross-sections of an oval shape or those with at least one defined angle to include obtuse, acute, and right angles can provide a shape in some situations. A substantially round shape may also be employed. Further, shapes specifically or custom-designed for the user may be provided.

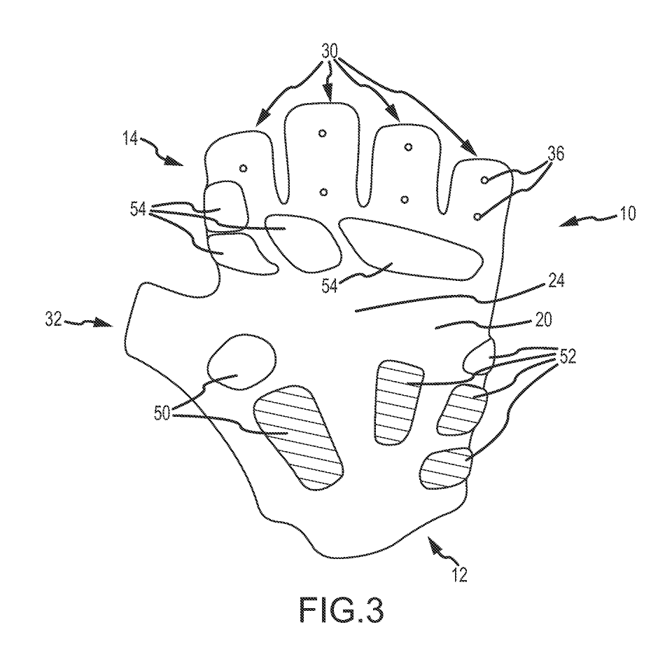

Another embodiment of the invention is depicted in FIG. 3, a top-view of the palmar-side of the glove 10. A glove 10 that fits over a human hand is shown with lower glove 12 portion and upper glove portion 14, the glove 10 comprising a main glove body 20 and a glove palm 24, a finger area 30 and thumb area 32. The glove 10 may be fitted with glove holes 36 or gaps, for example glove holes 36 as shown in FIG. 3 on the finger area 30. In other embodiments, the glove holes 36 may be fitted to the glove thumb area 32 and/or to the main glove body area 20. In this embodiment of the invention, the glove 10 fits over the hand of the user without need of a closure tab 40 which affixes to the closure tab receiving area 42, as featured in the embodiment of FIGS. 1A and 1B. The palmar-side of the glove 10 as shown in FIG. 3 depicts one or more pads located on the glove palm 24. The pads comprise thumb-area pads 50, side-area pads 52, and finger-area pads 54. The pads are deliberately and precisely positioned on the glove 10 to receive pressure, when gripping a hard object such as bicycle handlebars while bicycling, that is directed through the pads and substantially toward, and substantially received by, the boney regions of the hand, and furthermore, not over broad regions of the hand comprising the nerves of the hand.

In the embodiment of the invention of FIG. 3, the pads fitted to the glove 10 are of variable height and comprise thumb-area pads 50, side-area pads 52, and finger-area pads 54. In this embodiment of the invention, there are four pads of type finger-area pads 54, four pads of type side-area pads 52, and two pads of type thumb-area pads 50. Further, in this embodiment, each of the lowest four pads located in the lower glove area, shown with cross-hatching in FIG. 3, are of height about 4 mm. Three of these pads are of type side-area pads 52 and one is of type thumb-area pads 50. The remaining six pads, depicted in FIG. 3 without cross-hatching, one of type side-area pads 52, one of type thumb-area pads 50, and four of type finger-area pads 54, are of height about 2 mm.

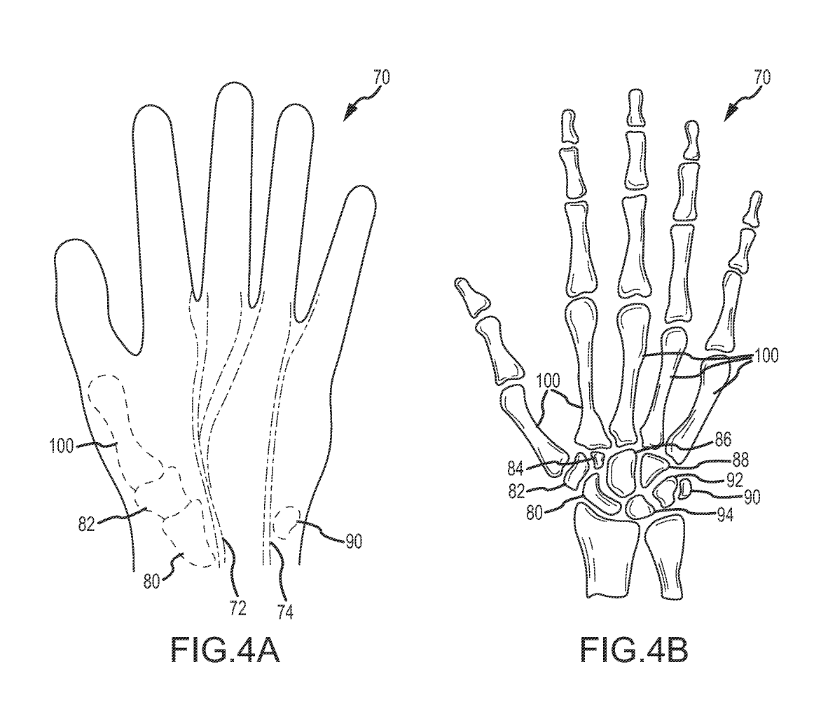

Referring now in detail to FIGS. 4A and 4B, schematic anatomical views of selected bones and nerves of the human hand from the palmar side are provided. Referring to FIG. 4A, a hand 70 is provided with median nerve 72 and ulnar nerve 74 depicted, and a metacarpal bone 100, the trapezium bone 82, the scaphoid bone 80, and the pisiform bone 90. In one preferred embodiment of the invention, the one or more pads are configured to not substantially overlap or cover one or more of the median nerve 72 and ulnar nerve 74, and to transfer force and/or pressure to one or more of the hamate bone 88, the trapezium bone 82, the scaphoid bone 80, and the pisiform bone 90. Referring to FIG. 4B, a hand 70 is provided including all metacarpal bones 100, the hamate bone 88, the trapezium bone 82, the scaphoid bone 80, the pisiform bone 90, the trapezoid bone 84, the capitate bone 86, the triquetrum bone 92, and the lunate bone 94. In another embodiment of the invention, the one or more pads are configured to not substantial overlap or cover one or more of the median nerve 72 and ulnar nerve 74, and transfer force and/or pressure to one or more of the bones of the hand, to include the bones depicted in FIG. 4B.

In one embodiment of the invention, the location of the pads is identified with reference to specific boney regions of the hand, so as to direct pressure and/or force substantially through the one or more pads and in-turn substantially through the one or more boney regions or bones of the hand. In one preferred embodiment, the boney regions of the hands that receive pressure and/or force comprise the pisoform, hamate, scaphoid, and trapezium bones, and maintains an open channel for one or more nerves of the hand.

To lend further clarity to the Detailed Description provided herein in the associated drawings, the following list of components and associated numbering are provided:

| Reference No. | Component |
|---|---|
| 10 | Glove |
| 12 | Lower Glove |
| 14 | Upper Glove |
| 20 | Main Glove Body |
| 24 | Glove Palm |
| 30 | Finger Area |
| 32 | Thumb Area |
| 36 | Glove Holes |
| 40 | Closure Tab |
| 42 | Closure Tab Receiving Area |
| 50 | Thumb-area Pads |
| 52 | Side-area Pads |
| 54 | Finger-area Pads |
| 56 | Channel |
| 60 | Pad Surface |
| 62 | Pad Edge |
| 64 | Pad Depression |
| 68 | Pad Plate |
| 70 | Hand |
| 72 | Median Nerve |
| 74 | Ulnar Nerve |
| 80 | Scaphoid Bone |
| 82 | Trapezium Bone |
| 84 | Trapezoid Bone |
| 86 | Capitate Bone |
| 88 | Hamate Bone |
| 90 | Pisiform Bone |
| 92 | Triquetrum Bone |
| 94 | Lunate Bone |
| 100 | Metacarpal Bone |

While various embodiment of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim:

1. A glove apparatus comprising:
a glove body comprising a palmar-side and a dorsal-side, the glove body constructed to cover at least a portion of a user's palm;
at least one pad comprising a first pad; and
wherein the first pad is configured to attach to the palmar-side of the glove body,
wherein the first pad is positioned on the palmar-side of the glove body to maintain a first gap between an edge of the first pad and an edge of the user's palm,
and wherein the first pad is configured to receive a gripping force imparted to the user's hand and transfer gripping force received to at least one of a carpal bone of the user's hand.

2. The apparatus of claim 1, wherein the at least one of a carpal bone of the user's hand is a bony pillar of the user's hand.

3. The apparatus of claim 2, wherein the bony pillar of the user's hand is selected from the group consisting of a hamate bone and a pisiform bone.

4. The apparatus of claim 1, wherein the at least one pad is a plurality of pads comprising the first pad and a second pad, the second pad positioned on the palmar-side of the glove body, and wherein the second pad is configured to receive a gripping force imparted to the user's hand and transfer gripping force received to at least one of a carpal bone of the user's hand.

5. The apparatus of claim 4, wherein the first pad and the second pad are configured to be in communication when a gripping force is imparted to the user's hand such that the first pad and the second pad, in combination, transfer respective gripping force received to at least a plurality of carpal bones of the user's hand.

6. The apparatus of claim 5, wherein the first pad and the second pad are configured to form a second gap between an edge of the first pad and an edge of the second pad when the user's hand is at least one of unflexed, relaxed and not gripping and wherein the first pad and the second pad are further configured to close the second gap when the user's hand is at least one of gripping and forming a fist.

7. The apparatus of claim 1, wherein the first pad is positioned not substantially over the user's median and ulnar neurovascular bundles.

8. The apparatus of claim 4, wherein the second pad is positioned not substantially over the user's median and ulnar neurovascular bundles.

9. The device of claim 1, wherein the one or more pads are removably attached to the palmar-side of the glove body.

10. The device of claim 1, wherein the first pad is configured of cross-sectional shape selected from the group consisting of a rectangle, a dome, an oval, and a dome configured with one or more indentations.

11. The device of claim 1, wherein the glove body is further constructed to cover at least a portion of the user's palm, wherein the dorsal-side is comprised of a material selected from the group consisting of a nylon material, an elastic textile material, and mesh material and the palmar-side is comprised of a material selected from the group consisting of perforated synthetic leather, perforated leather, breathable synthetic leather, and breathable leather, polyester, wool, and insulating material.

12. The apparatus of claim 7, wherein the first pad is located not more than 2 mm from the user's median and ulnar neurovascular bundles.

13. The apparatus of claim 4, wherein the plurality of pads comprise no more than 50 percent of the surface area of the palmar-side of the glove.

14. A glove apparatus comprising:
a glove body comprising a palmar-side and a dorsal-side, the glove body constructed to cover at least a portion of a user's palm;
at least one pad comprising a first pad; and
wherein the first pad is positioned on the palmar-side of the glove body and positioned not substantially over the user's median and ulnar nerves, and positioned to cover one or more of the metacarpal bones,
and wherein the first pad is configured to receive a gripping force imparted to the user's hand and transfer gripping force received to at least one of a hamate bone and a pisiform bone of the user's hand.

15. The apparatus of claim 14, wherein the at least one pad is a plurality of pads comprising the first pad and a second pad, the second pad positioned on the palmar-side of the glove body, and wherein the second pad is configured to receive a gripping force imparted to the user's hand and transfer gripping force received to at least one of a carpal bone of the user's hand.

16. The apparatus of claim 15, wherein the first pad and the second pad are configured to be in communication when a gripping force is imparted to the user's hand such that the first pad and the second pad, in combination, transfer respective gripping force received to at least a plurality of carpal bones of the user's hand.

17. The apparatus of claim 16, wherein the first pad and the second pad are configured to form a gap between an edge of the first pad and an edge of the second pad when the user's hand is at least one of unflexed, relaxed and not gripping and wherein the first pad and the second pad are further configured to close the second gap when the user's hand is at least one of gripping and forming a fist.

18. The device of claim 14, wherein the first pad is configured of cross-sectional shape selected from the group consisting of a rectangle, a dome, an oval, and a dome configured with one or more indentations.

19. A glove apparatus comprising:
a glove body comprising a palmar-side and a dorsal-side, the glove body constructed to cover at least a portion of a user's palm;
at least one pad comprising a first pad; and
wherein the first pad is positioned on the palmar-side of the glove body and adjacent to but not substantially over the user's ulnar neurovascular bundle,
and wherein the first pad is configured to receive a gripping force imparted to the user's hand and transfer gripping force received to at least one of a hamate bone and a pisiform bone of the user's hand.

20. The apparatus of claim 19, wherein the at least one pad is a plurality of pads comprising the first pad and a second pad, the second pad positioned on the palmar-side of the glove body, and wherein the second pad is configured to receive a gripping force imparted to the user's hand and transfer gripping force received to at least one of a carpal bone of the user's hand.
